(19) United States
(12) Patent Application Publication
(10) Pub. No.: US 2025/0040935 A1
(43) Pub. Date: Feb. 6, 2025

(54) FABRICATION OF RADIOPAQUE RESORBABLE HYDROGEL BEADS VIA MICROFLUIDICS FOR CATHETER EMBOLIZATION APPLICATIONS

(71) Applicant: Saint Louis University, St. Louis, MO (US)

(72) Inventors: Silviya Petrova Zustiak, St. Louis, MO (US); Andrew Hall, Weldon Springs, MO (US); Keith Pereira, St. Louis, MO (US)

(21) Appl. No.: 18/777,269

(22) Filed: Jul. 18, 2024

Related U.S. Application Data
(60) Provisional application No. 63/514,199, filed on Jul. 18, 2023.

(51) Int. Cl. A61B 17/12; A61L 24/00; A61L 24/04

(52) U.S. Cl. CPC A61B 17/1219; A61B 17/12031; A61B 17/12109; A61L 24/0015; A61L 24/0031; A61L 24/046; A61B 2017/1205; A61L 2300/102; A61L 2300/416; A61L 2300/43; A61L 2300/45; A61L 2300/622; A61L 2430/36

(57) ABSTRACT

Disclosed are compositions and methods of treating prostate cancer. The compositions include biocompatible and resorbable hydrogel embolization microspheres that contain a contrast agent, a hormone therapy, and a chemotherapeutic agent.

### CROSS-REFERENCE TO RELATED APPLICATIONS

This application claims the benefit of priority to U.S. Application Ser. No. 63/514,199 filed on Jul. 18, 2023, the contents of which are herein incorporated by reference in their entirety.

### BACKGROUND

The present disclosure relates generally to medicine. More particularly, the present disclosure is directed to compositions and methods for treating prostate cancer.

Prostate cancer (PCa) remains the most common cancer in men and is a major cause of cancer-related death. In 2018, an estimated 164,690 new cases of PCa were detected with about 29,430 attributable to this cancer. Following PCa diagnosis, patients face a multitude of options: active surveillance, observation/watchful waiting, prostatectomy, radiotherapy, cryosurgery, and high intensity focused ultrasound and focal therapy. In early stages, radiation and removal of the entire prostate (prostatectomy) are recommended. However, radiation is associated with diarrhea, bloody urine/stools, burning sensation during urination, and erectile problems. Prostatectomy is the standard of care, but is a major surgery associated with incontinence and erectile dysfunction. About 15% of patients regret seeking treatment. Options for patients in later stages include chemotherapy and hormonal therapy. However, drugs and hormones must currently be delivered to the entire body via the blood stream, causing diarrhea, enlarged/tender breasts, nausea, hot flashes, loss of libido, and erectile dysfunction. Prostate-specific antigen (PSA) screening has helped detect lower-stage tumors at a younger age, making overtreatment a fundamental problem. Younger patients are interested in preserving not only continence but also sexual function, both important in quality of life.

Transarterial embolization is used to treat hepatocellular cancer. Transarterial embolization is performed by injecting a chemotherapeutic drug selectively into an artery feeding the target tumoral nodules, followed by embolization of the same vessel to obtain a synergistic effect of either cytotoxic activity and ischemia. Chemoembolization is a treatment that has been successful in treating liver cancer and works by injecting hydrogel microspheres, loaded with chemotherapy drugs, directly into the hepatic artery in the liver where the tumor is located. Current chemoembolization treatments include separately injecting a contrast agent prior to bead embolization to show the clinician where the beads will end up. Transarterial chemoembolization suffers from drawbacks such as the lack of standardization and unpredictability of outcomes and release of the injected drug into systemic circulation. Off-target embolization is another significant risk and clinicians have no way of assessing how many beads made it to the target location and how many were lodged into off-target locations (and which off-target locations). The development of non-absorbable embolic microspheres containing cytotoxic agents, referred to as drug-eluting beads, are able to simultaneously exert both of the therapeutic components of transarterial chemoembolization, either drug-carrier function and embolization. The drug-eluting beads are composed of a hydrophilic, ionic polymer that can bind anthracyclines via an ion exchange mechanism. The risk of systemic drug release is minimal due to both high-affinity carrier activity of drug-eluting beads and the absence of a time interval between injection and embolization. DC Bead LUMI™ and HEPASHPHERE™ microspheres have been commercialized for the local treatment of malignant hypervascularized tumor(s) in the liver and to embolize the vessels supplying malignant colorectal cancer metastasized to the liver. The DC Bead LUMI™ are 70-150 μm microspheres that consist of a biocompatible, sulphonate-modified, N-Fil hydrogel. Other DC Beads are available in 3 sizes that range from 100 μm to 500 km. Traditional contrast agents are absorbed in the DC Bead outer regions, where the contrast agent elutes within minutes to hours following embolization injection. While helping with embolization injection (specifically using cone-beam CT imaging), follow up imaging to assess off-target embolization is not possible. Another approach adds barium sulfate particles on the surface of hydrogel beads, which again limits the follow up imaging.

While chemoembolization for liver is used clinically with better efficacy and lower side effects compared to systemic chemotherapy, such chemoembolization for prostate cancer does not exist. Prostate cancer is typically treated using hormonal treatment (e.g. apalutamide) via oral administration or androgen depravation therapy (ADT) to reduce prostate tumor growth. ADT can be effective as a monotherapy or in combination with the other treatments discussed. Estrogens, gonadotropin releasing hormone agonists, androgen receptor blockers and other medications can be used for ADT. Chemotherapy (e.g. docetaxel) to reduce prostate tumor growth is currently given systemically. Chemotherapy, while largely effective against other forms of cancer, is not commonly considered to be as effective against prostate cancer. Cabazitaxel is a second-line chemotherapy drug used after failed treatments of docetaxel. While cabazitaxel can treat paclitaxel- and docetaxel-resistant tumors, it presents high risk of adverse blood conditions like neutropenia, leukopenia, and anemia. In addition to the severe risks posed by docetaxel and cabazitaxel specifically, systemic chemotherapy commonly causes nausea, vomiting, diarrhea, stomatitis, and other side effects that drastically reduce the patient's quality of life during treatment. Systemic delivery of hormone and chemo therapies result in severe side-effects.

Accordingly, there exists a continuing need for developing new compositions and methods for treating prostate cancer. The present disclosure provides radiopaque microspheres for delivery of hormonal and chemotherapeutic agents and embolization for treating prostate cancer. Advantageously, the microspheres are resorbable with a fully integrated radiopaque material such that the radiopacity at any point in time functions as an indicator of the level of resorption of the microspheres, and thus, allow for repeated administration.

### BRIEF DESCRIPTION

The present disclosure relates generally to compositions and methods for treating prostate cancer. In particular, the present disclosure relates to resorbable radiopaque microspheres that provide embolization while also delivering hormonal and chemotherapeutic agents for treating prostate cancer.

In one aspect, the present disclosure is directed to an embolization composition comprising: a plurality of biocompatible and resorbable polymer hydrogel microspheres comprising a contrast agent; a hormonal therapy; and a chemotherapeutic; and a carrier.

In one aspect, the present disclosure is directed to a biocompatible and resorbable polymer hydrogel microspheres comprising a contrast agent; a hormonal therapy; and a chemotherapeutic.

In one aspect, the present disclosure is directed to a method of treating prostate cancer in a subject in need thereof, the method comprising: injecting into a blood vessel of the subject an embolization composition comprising a plurality of biocompatible and resorbable polymer hydrogel microspheres, the biocompatible and resorbable polymer hydrogel microspheres comprising a contrast agent; a hormonal therapy; and a chemotherapeutic; and a carrier.

### DESCRIPTION OF THE DRAWINGS

The patent or application file contains at least one drawing executed in color. Copies of this patent or patent application publication with color drawing(s) will be provided by the Office upon request and payment of the necessary fee.

The disclosure will be better understood, and features, aspects and advantages other than those set forth above will become apparent with consideration is given to the following detailed description thereof. Such detailed description makes reference to the following drawings, wherein:

FIG. 1 is an illustration depicting the mechanism of PEG hydrogel formation and capture of solutes during gelation.

FIGS. 2A and 2B are graphs depicting docetaxel release from PEG gels (FIG. 2A) and tumor volume (FIG. 2B) of PC3 cells xenograft in mice upon treatment with beads+docetaxel and beads only.

FIGS. 3A-3D depict imaging of barium sulfate loaded microspheres. FIG. 3A is an optical microscope image. (Scale bar 100 μm). FIG. 3B depicts a single slice microCT image. (Scale bar 200 μm). FIG. 3C depicts a single slice axial view cone beam CT image of whole mouse xenograft model with microspheres injected at the tumor surface. (Scale bar 5 mm). FIG. 3D depicts a single slice microCT image of spheres in an excised tumor. (Scale bar 100 μm).

FIG. 4 is a schematic illustrating the fabrication of chemotherapy loaded barium sulfate-PEG hydrogels using 4-arm PEG-Ac and PEG-diSH. The 4-arm PEG-Ac and PEG-diSH polymers form a crosslinking network that encapsulates the barium sulfate contrast agent, and chemotherapy.

FIG. 5 is a schematic illustrating an exemplary T-junction to create microspheres.

FIG. 6 is a photograph depicting an exemplary T-junction microfluidic device for fabricating microspheres.

FIG. 7 are photographs depicting the effect of ethanol and DMSO solvents for drugs on the settling of barium sulfate with and without drugs (docetaxel and bicalutamide).

FIGS. 8A and 8B are photographs depicting opaqueness of PEG hydrogels containing barium sulfate with drugs in ethanol (“Eth”) solvent and DMSO solvent. FIG. 8A depicts microscopic images of hydrogels (Scale bar=200 μm). FIG. 8B depicts macroscopic images of hydrogels (Scale bar=1 cm).

FIGS. 9A-9C depict the effect of drug loading on 1 μm BaSO₄ microsphere opacity, size, and swelling. FIG. 9A depicts the opacity of barium microspheres with and without drugs loaded. Scale bar=200 μm. FIG. 9B is a histogram depicting microsphere size pre- and post-wash. (n=200). FIG. 9C is a graph depicting the swelling ratio of 1 μm BaSO₄ microspheres with and without drugs loaded. (p-value=0.0002).

FIGS. 10A-10C depict the effect of barium sulfate particle size on microsphere opacity, size, and swelling. FIG. 10A depicts the opacity of microspheres with 0.1 μm and 1 μm BaSO₄ particles pre- and post-wash. Scale bar=200 μm. FIG. 10B is a histogram depicting size of microsphere with 0.1 μm and 1 μm BaSO₄ particles pre- and post-wash. (n=200). FIG. 10C is a graph depicting the swelling ratio of microsphere with 0.1 μm and 1 μm BaSO₄ particles pre- and post-wash.

FIG. 11 depicts the degradation and imageability of microspheres containing 0.1 μm and 1 μm BaSO₄ particles over time.

FIGS. 12A-12C depict the effect of BaSO₄ on release of bicalutamide and docetaxel loaded in the same gel. FIG. 12A depicts the cumulative mass release of bicalutamide and docetaxel from different hydrogels. FIG. 12B depicts the fractional release of bicalutamide and docetaxel as a function of the square root of time. FIG. 12C depicts the effective diffusion coefficient of bicalutamide and docetaxel. (n=3).

FIGS. 13A-13C depict the effect of BaSO₄ on the release of docetaxel and apalutamide. FIG. 13A depicts the cumulative mass release of docetaxel and apalutamide. FIG. 13B depicts the fractional release of docetaxel and apalutamide. FIG. 13C depicts the effective diffusion coefficient of docetaxel and apalutamide. (n=3).

FIGS. 14A-14C depict the effect of BaSO₄ on the release of docetaxel and apalutamide from microspheres. FIG. 14A depicts the cumulative mass release of docetaxel and apalutamide. FIG. 14B depicts the fractional release of docetaxel and apalutamide. FIG. 14C depicts the effective diffusion coefficient of docetaxel and apalutamide. (n=3).

FIGS. 15A and 15B are graphs depicting the storage and loss modulus of barium sulfate-loaded hydrogels with and without drugs. FIG. 15A depicts storage modulus and loss modulus. FIG. 15B depicts the average and standard deviation of storage modulus.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

While the present disclosure is susceptible to various modifications and alternative forms, exemplary embodiments thereof are shown by way of example in the drawings and are herein described in detail. It should be understood, however, that the description of exemplary embodiments is not intended to limit the disclosure to the particular forms disclosed, but on the contrary, the intention is to cover all modifications, equivalents and alternatives falling within the scope of the disclosure as defined by the embodiments above and the claims below. Reference should therefore be made to the embodiments above and claims below for interpreting the scope of the present disclosure.

Unless defined otherwise, all technical and scientific terms used herein have the same meaning as commonly understood by one of skill in the art to which the invention pertains. Although any methods and materials similar to or equivalent to those described herein can be used in the practice or testing of the present disclosure, the preferred methods and materials are described herein. Moreover, reference to an element by the indefinite article “a” or “an” does not exclude the possibility that more than one element is present, unless the context clearly requires that there be one and only one element. The indefinite article “a” or “an” thus usually includes “at least one.” The term “about” means up to ±10%.

As used herein, “susceptible” and “at risk” refer to having little resistance to a certain disease, disorder or condition, including being genetically predisposed, having a family history of, and/or having symptoms of the disease, disorder or condition.

As used herein, “a subject in need thereof” (also used interchangeably with “a patient in need thereof”), as it relates to the therapeutic uses herein, is one identified to require or desire medical intervention. Because some of the method embodiments of the present disclosure are directed to specific subsets or subclasses of identified subject (that is, the subset or subclass of subject “in need” of assistance in addressing one or more specific conditions noted herein), not all subject will fall within the subset or subclass of subject in need of treatment described herein. An effective amount is that amount of an agent necessary to inhibit the pathological diseases and disorders herein described. When at least one additional therapeutic agent is administered to a subject, such additional agents may be administered sequentially, concurrently, or simultaneously, in order to obtain the benefits of the agents. The term patient includes vertebrate animals, and preferably is a human patient.

In one aspect, the present disclosure is directed to an embolization composition comprising: a plurality of biocompatible and resorbable polymer hydrogel microspheres comprising a contrast agent; a hormonal therapy; and a chemotherapeutic; and a carrier.

Suitable polymer hydrogel microspheres include polyethylene glycol hydrogel microspheres, polyacrylate hydrogel microspheres, polyvinylpyrrolidone hydrogel microspheres, and combinations thereof. Particularly suitable polymer hydrogel microspheres are polyethylene glycol (PEG)-based microspheres prepared via crosslinking a polymer and/or a crosslinker via Michael-type addition, photopolymerization or other click chemistry methods. Suitable PEG-based polymer for preparing the biocompatible and resorbable polymer hydrogel microspheres include poly(ethylene glycol)-di-acrylate (PEG-DA), multi-arm poly(ethylene glycol)-acrylate (PEG-Ac), poly(ethylene glycol) divinyl sulfone (PEG-diVS), multi-arm poly(ethylene glycol) vinyl sulfone (PEG-VS), poly(ethylene glycol)-di-methacrylate (PEG-DMA), multi-arm poly(ethylene glycol)-methacrylate (PEG-MAc), poly(ethylene glycol)-di-allyl ether (PEG-diAE), multi-arm poly(ethylene glycol)-allyl ether (PEG-AE), poly(ethylene glycol)-di-vinyl ether (PEG-diVE), multi-arm poly(ethylene glycol)-vinyl ether (PEG-VE), poly(ethylene glycol)-di-maleimide (PEG-diMI), multi-arm poly(ethylene glycol)-maleimide (PEG-MI), poly(ethylene glycol)-di-norborene, multi-arm poly(ethylene glycol)-norborene, poly(ethylene glycol)-di-vinyl carbonate, multi-arm poly(ethylene glycol)-vinyl carbonate, polyethylene glycol oligofumarate, and combinations thereof. Suitable crosslinkers include thiol-functionalized molecules such as poly(ethylene glycol)-dithiol (PEG-diSH), multi-arm polyethylene thiol (PEG-SH), degradable poly(ethylene glycol)dithiol-based crosslinkers (labelled as PEG-diester-dithiol crosslinkers; see Table 1), dithiothreitol (DTT), glycol dimercaptoacetate (GDMA), glyceryldithioglycolate (GDT), glycol di(3-mercaptopropionate) (GDMP), tetraethylene glycol dithiol (TEGDT), 2,2′-(ethylenedioxy) diethanethiol (EDDT), 2-amino butane dithiol (DTBA), or combinations thereof. For simplicity, the resultant hydrogel microspheres upon crosslinking the polymer and/or crosslinker are denoted herein as polyethylene glycol (PEG).

As used herein, the term “biocompatible” is used according to its ordinary meaning as is understood by one of ordinary skill in the art to mean compatibility with living tissue or a living system by not being toxic, injurious, or physiologically reactive and not causing immunological rejection of the biocompatible and resorbable polymer hydrogel microspheres of the present disclosure.

As used herein, the term “degradable” is used according to its ordinary meaning as is understood by one of ordinary skill in the art to mean to break down. Suitably, the biocompatible and resorbable polymer hydrogel microspheres degrade at a rate ranging from about 6 hours to about 8 weeks. In an exemplary embodiment, the biocompatible and resorbable polymer hydrogel microspheres degrade at approximately the same rate. In an exemplary embodiment, biocompatible and resorbable polymer hydrogel microspheres degrade at different rates. In an exemplary embodiment, one set of biocompatible and resorbable polymer hydrogel microspheres having a first degradation rate is combined with another set of biocompatible and resorbable polymer hydrogel microspheres having a degradation rate that is different than the first degradation rate to create a mixture of biocompatible and resorbable polymer hydrogel microspheres having different degradation rates.

Suitable contrast agents include barium sulfate, iodine, gold, zirconium oxide, bismuth, gold, and combinations thereof. The contrast agent particle size contained by the biocompatible and resorbable polymer hydrogel microspheres ranges in average diameter from about 0.1 μm to about 3 μm.

Suitable hormone therapies include androgen receptor antagonists, including non-steroidal androgen receptor antagonist including bicalutamide; apalutamide; darolutamide; enzalutamide; flutamide; nilutamide and combinations thereof, and steroidal androgen receptor antagonists including cyproterone acetate, medroxyprogesterone acetate and megestrol acetate, and combinations thereof.

Suitable chemotherapeutics include docetaxel, cabazitaxel, mitoxantrone, estramustine, and combinations thereof.

Suitably, the biocompatible and resorbable polymer hydrogel microspheres release the hormone therapy at a rate ranging from about 5 minutes to about 1 week. In an exemplary embodiment, the biocompatible and resorbable polymer hydrogel microspheres release the hormone therapy at approximately the same rate. In an exemplary embodiment, biocompatible and resorbable polymer hydrogel microspheres release the hormone therapy at a different rate. In an exemplary embodiment, one set of biocompatible and resorbable polymer hydrogel microspheres that release the hormone therapy at a first rate is combined with another set of biocompatible and resorbable polymer hydrogel microspheres that release the hormone therapy at a rate that is different than the first rate to create a mixture of biocompatible and resorbable polymer hydrogel microspheres having different rates of releasing the hormone therapy.

Suitably, the biocompatible and resorbable polymer hydrogel microspheres release the chemotherapy at a rate ranging from about 5 minutes to about 1 week. Without being bound by theory, as the microspheres are administered, an initial “burst” release of the hormone and/or chemotherapeutic can occur such as, for example, in the first about 24 hours to about 48 hours. As the microspheres begin degrading, hormone and/or chemotherapeutic is released from the degrading microspheres. In an exemplary embodiment, the biocompatible and resorbable polymer hydrogel microspheres release the chemotherapy at approximately the same rate. In an exemplary embodiment, biocompatible and resorbable polymer hydrogel microspheres release the chemotherapy at a different rate. In an exemplary embodiment, one set of biocompatible and resorbable polymer hydrogel microspheres that release the chemotherapy at a first rate is combined with another set of biocompatible and resorbable polymer hydrogel microspheres that release the chemotherapy at a rate that is different than the first rate to create a mixture of biocompatible and resorbable polymer hydrogel microspheres having different rates of releasing the chemotherapy.

The rate of releasing the hormone and the rate of releasing the chemotherapy are controlled by varying at least one of the microsphere degradation rate, the size of the microsphere containing the hormone and the chemotherapy, the polyethelene glycol polymer concentration, the molecular weight of the polymer used to fabricate the microspheres, the polymer type, the drug type, and combinations thereof.

The biocompatible and resorbable polymer hydrogel microspheres suitably have an average diameter ranging from about 50 microns to about 1000 microns, more typically in the range of about 100 microns to about 300 microns, of about 70 microns to about 150 microns, of about 300 microns to about 500 microns, and of about 500 microns to about 700 microns. Average diameter of the biocompatible and resorbable polymer hydrogel microspheres can be determined by measuring individual hydrogel microspheres in micrographic images of the biocompatible and resorbable polymer hydrogel microspheres, for example.

The embolization composition further includes a carrier. The carrier functions as a medium to administer the plurality of biocompatible and resorbable polymer hydrogel microspheres. Suitably, the carrier is a pharmaceutically acceptable carrier. As understood by those skilled in the art, pharmaceutically acceptable carriers, and, optionally, other therapeutic and/or prophylactic ingredients must be “acceptable” in the sense of being compatible with the other ingredients of the formulation and not be harmful to the recipient thereof. Suitably, the carrier is a buffer. Suitable buffers include water, saline, isotonic saline, phosphate buffered saline, sodium acetate, glycylglycine, HEPES (4-(2-hydroxyethyl)-1-piperazineethanesulfonic acid), sodium phosphate, dimethyl sulfoxide, ethanol, thiethanolamine, and a combination thereof. The embolization composition of the present disclosure can be administered to animals, preferably to mammals, and in particular to humans as therapeutics per se, as mixtures with one another or in the form of pharmaceutical preparations, and which as active constituent contains an effective dose of the active agents (e.g., hormone therapy and chemotherapy), in addition to customary pharmaceutically innocuous excipients and additives.

The therapy can further include other components such as surfactants, preservatives, and excipients. Suitable surfactants fatty acid esters and alcohols, and polyoxyethylene sorbitol fatty acid esters. Amounts will generally range from about 0.001 and about 4% by weight of the formulation. Pharmaceutically acceptable preservatives include, for example, phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) and mixtures thereof. The preservative can be present in concentrations ranging from about 0.1 mg/ml to about 20 mg/ml, including from about 0.1 mg/ml to about 10 mg/ml. The use of a preservative in pharmaceutical compositions is well-known to those skilled in the art. For convenience reference is made to Remington: The Science and Practice of Pharmacy, 19th edition, 1995. Formulations can include suitable buffers such as sodium acetate, glycylglycine, HEPES (4-(2-hydroxyethyl)-1-piperazineethanesulfonic acid) and sodium phosphate. Excipients include components for tonicity adjustment, antioxidants, and stabilizers as commonly used in the preparation of pharmaceutical formulations. Other inactive ingredients include, for example, L-histidine, L-histidine monohydrochloride monohydrate, sorbitol, polysorbate 80, sodium citrate, sodium chloride, and EDTA disodium.

The embolization composition of the present disclosure is useful in methods for treating a solid cancer tumor. The embolization composition of the present disclosure is particularly useful in methods for treating a solid cancer tumor having blood vessels that directly supply the solid cancer tumor. The embolization composition of the present disclosure is administered into a blood vessel that supplies blood to the solid cancer tumor. Because the biocompatible and resorbable polymer hydrogel microspheres of the embolization composition includes a contrast agent, the embolization composition can be observed during and after administration into the blood vessel. The biocompatible and resorbable polymer hydrogel microspheres of the embolization composition also occlude the blood vessels that supply the solid cancer tumor to block or reduce oxygen supply to the solid cancer tumor. The biocompatible and resorbable polymer hydrogel microspheres of the embolization composition further “trap” blood flow, restricting and concentrating the hormone therapy and chemotherapy in the solid cancer tumor to improve efficacy and reduce side effects of the hormone therapy and chemotherapy by preventing the hormone therapy and chemotherapy from being washed into the systemic circulation causing side-effects at distant sites. The same biocompatible and resorbable polymer hydrogel microsphere functions to deliver a hormone therapy and a chemotherapy. The same biocompatible and resorbable polymer hydrogel microsphere also incorporates a contrast agent, and thus, functions to allow imaging of the biocompatible and resorbable polymer hydrogel microspheres during administration to permit localization and following degradation of the biocompatible and resorbable polymer hydrogel microspheres. Suitable solid cancer tumors that can be treated using the embolic composition include prostate cancer, hepatoma, hepatocellular carcinoma (HCC), liver metastasis, cholangiomas, neuroendocrine tumors, GIST liver metastasis, and renal cancer. Prostate cancer is particularly suitable for treatment using the embolic composition of the present disclosure.

In one aspect, the present disclosure is directed to a method of treating a subject having or suspected of having prostate cancer. The method includes injecting into a blood vessel of the subject a plurality of biocompatible and resorbable polymer hydrogel microspheres that comprise: a contrast agent; a hormonal therapy; and a chemotherapeutic.

Suitably, the method includes injecting the plurality of biocompatible and resorbable polymer hydrogel microspheres directly into a blood vessel that supplies the prostate cancer tumor.

Suitable methods for administration of formulations of the present disclosure are by intravenous (IV) routes and the formulations administered ordinarily include effective amounts of product in combination with acceptable diluents, carriers and/or adjuvants. Standard diluents such as human serum albumin are contemplated for pharmaceutical compositions of the disclosure, as are standard carriers as described herein.

As used herein, an “effective amount”, a “therapeutically effective amount”, a “prophylactically effective amount” and a “diagnostically effective amount” is the amount of the therapy of the present disclosure needed to elicit the desired biological response following administration of the therapy. Suitable dosage for use in the methods of the present disclosure will depend upon a number of factors including, for example, age and weight of a subject, severity of the prostate cancer, nature of a composition, route of administration and combinations thereof. Ultimately, a suitable dosage can be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, “treating” (or “treat” or “treatment”) refers to processes involving a slowing, interrupting, arresting, controlling, stopping, reducing, or reversing the progression or severity of an existing symptom, disorder, condition, or disease, but does not necessarily involve a total elimination of all disease-related symptoms, conditions, or disorders associated with administration of the therapy. Treatment is intended to induce a desired therapeutic effect, the desired therapeutic effect being clinical remission, clinical response, remission, healing and/or symptomatic remission.

The method can further include imaging the subject to determine location of the plurality of biocompatible and resorbable polymer hydrogel microspheres. The method can further include imaging the subject to determine degradation status of the plurality of biocompatible and resorbable polymer hydrogel microspheres. Suitable methods for imaging the subject to determine location and/or degradation status include microCT; clinical CT; X-ray; and combinations thereof. Determining degradation status of the biocompatible and resorbable polymer hydrogel microspheres is useful to determine whether another administration of the plurality of biocompatible and resorbable polymer hydrogel microspheres should be performed.

Tumor growth can be determined using methods known in the art such as magnetic resonance imaging (MRI), for example.

The method can further include administering at least one of the hormone therapy, the chemotherapy, and a combination thereof, prior to administration of the plurality of the biocompatible and resorbable polymer hydrogel microsphere.

In another aspect, the present disclosure is directed to a biocompatible and resorbable polymer hydrogel microsphere that includes a contrast agent, a hormone therapy, and a chemotherapeutic.

Suitable polymer hydrogel microspheres include polyethylene glycol hydrogel microspheres, polyacrylate hydrogel microspheres, polyvinylpyrrolidone hydrogel microspheres, and combinations thereof, as described herein. Particularly suitable polymer hydrogel microspheres are polyethylene glycol (PEG)-based microspheres prepared via crosslinking a polymer and/or a crosslinker via Michael-type addition, photopolymerization or other click chemistry methods, as described herein. Suitable PEG-based polymer for preparing the biocompatible and resorbable polymer hydrogel microspheres include poly(ethylene glycol)-di-acrylate (PEG-DA), multi-arm poly(ethylene glycol)-acrylate (PEG-Ac), poly(ethylene glycol) divinyl sulfone (PEG-diVS), multi-arm poly(ethylene glycol) vinyl sulfone (PEG-VS), poly(ethylene glycol)-di-methacrylate (PEG-DMA), multi-arm poly(ethylene glycol)-methacrylate (PEG-MAc), poly(ethylene glycol)-di-allyl ether (PEG-diAE), multi-arm poly(ethylene glycol)-allyl ether (PEG-AE), poly(ethylene glycol)-di-vinyl ether (PEG-diVE), multi-arm poly(ethylene glycol)-vinyl ether (PEG-VE), poly(ethylene glycol)-di-maleimide (PEG-diMI), multi-arm poly(ethylene glycol)-maleimide (PEG-MI), poly(ethylene glycol)-di-norborene, multi-arm poly(ethylene glycol)-norborene, poly(ethylene glycol)-di-vinyl carbonate, multi-arm poly(ethylene glycol)-vinyl carbonate, polyethylene glycol oligofumarate, and combinations thereof, as described herein. Suitable crosslinkers include thiol-functionalized molecules such as poly(ethylene glycol)-dithiol (PEG-diSH), multi-arm polyethylene thiol (PEG-SH), degradable poly(ethylene glycol)dithiol-based crosslinkers (labelled as PEG-diester-dithiol crosslinkers; see Table 1), dithiothreitol (DTT), glycol dimercaptoacetate (GDMA), glyceryldithioglycolate (GDT), glycol di(3-mercaptopropionate) (GDMP), tetraethylene glycol dithiol (TEGDT), 2,2′-(ethylenedioxy) diethanethiol (EDDT), 2-amino butane dithiol (DTBA), and combinations thereof, as described herein. For simplicity, the resultant hydrogel microspheres upon crosslinking the polymer and/or crosslinker are denoted herein as polyethylene glycol (PEG), as described herein.

Suitable contrast agents include barium sulfate, iodine, gold, zirconium oxide, bismuth, and combinations thereof, as described herein. The contrast agent particle size contained by the biocompatible and resorbable polymer hydrogel microspheres ranges from about 0.1 μm to about 3 μm in average diameter, as described herein.

Suitable hormone therapies include androgen receptor antagonists, including non-steroidal androgen receptor antagonist including bicalutamide, apalutamide, darolutamide, enzalutamide, flutamide, nilutamide, and combinations thereof, and steroidal androgen receptor antagonists including cyproterone acetate, medroxyprogesterone acetate, and megestrol acetate, and combinations thereof, as described herein.

Suitable chemotherapeutics include docetaxel, cabazitaxel, mitoxantrone, estramustine, and combinations thereof, as described herein.

The biocompatible and resorbable polymer hydrogel microsphere include an average diameter ranging from about 50 μm to about 700 μm, including about 70 μm to about 150 μm, of about 100 μm to about 300 μm, of about 300 μm to about 500 μm, and of about 500 μm to about 700 μm.

A particularly suitable method for making the biocompatible and resorbable polymer hydrogel microspheres is by electrospraying. In an exemplary embodiment, a hydrogel precursor solution containing a contrast agent is prepared using a polymer (e.g., 4-arm PEG-Ac), a contrast agent, and a crosslinker as described below. The solution is transferred to a hypodermic syringe connected to a needle. The electrospraying setup includes a mechanical syringe pump, a direct current high voltage generator connected to the needle tip, and a grounded collector. Solution droplets are collected in a grounded oil bath, allowed to gel, collected, and washed with PBS to remove oil.

The biocompatible and resorbable polymer hydrogel microspheres can then be stored at 4° C. or −80° C. in a hydrated state. The biocompatible and resorbable polymer hydrogel microspheres can also be lyophilized for storage.

To load the biocompatible and resorbable polymer hydrogel microspheres with hormone therapies and chemotherapeutics, the biocompatible and resorbable polymer hydrogel microspheres are lyophilized and then incubated in a concentrated solution of the hormone therapies and chemotherapeutics to load the biocompatible and resorbable polymer hydrogel microspheres with hormone therapies and chemotherapeutics.

Particularly suitable degradable crosslinkers include those in Table 1, below:

TABLE 1
Structures, abbreviations, and pKₐ values of crosslinkers of biocompatible and
biodegradable PEG-based dithiol crosslinkers. All crosslinkers have a molecular weight
of 3.4 kDa and are water soluble.

| Crosslinker | | | |
|---|---|---|---|
| Name | Abbreviation | Chemical Structure | Thiol pKₐ |
| PEG-dithiol | PEG-diSH | 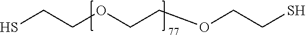 | ~10.5- 10.7 [44] |
| PEG- diester- dithiol-2 | PEG-DD2 | 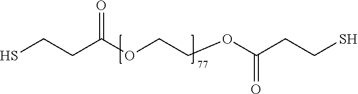 | ~9.4- 9.6 [44] |
| PEG- diester- dithiol-2- methyl | PEG-DD2M | 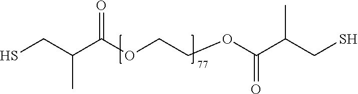 | ~9.4- 9.6 [44] |
| PEG- diester- dithiol-1 | PEG-DD1 | 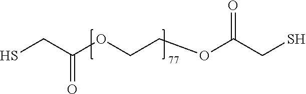 | ~7.9- 8.1 [44, 47] |
| PEG- diester- dithiol-1- methyl | PEG-DD1M | 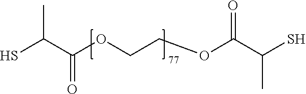 | ~7.9- 8.1 [44, 47] |
| PEG- diester- dithiol-1- phenyl | PEG-DD1P | 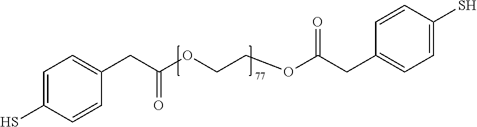 | ~6.5 [52, 53] |
| PEG- diester- dithiol-2- phenyl | PEG-DD2P | 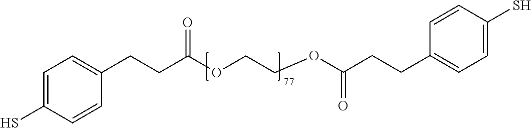 | ~6.5 [52, 53] |

Embodiments of the present disclosure are described in the following examples.

### EXAMPLES

Embodiments of the present disclosure are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example 1

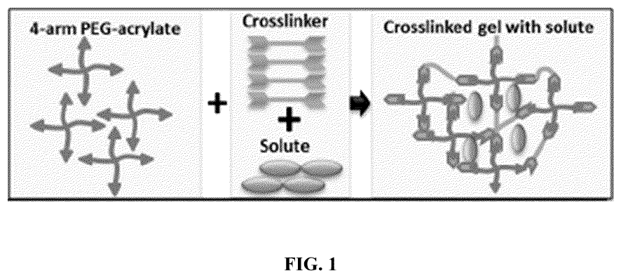

PEG hydrogel formation by electrospraying. Microsphere beads were fabricated using polyethylene glycol (PEG). PEG is biocompatible, inert and approved by the Food and Drug Administration for biomedical use. As illustrated in FIG. 1, PEG hydrogel beads form and capture solutes during gelation by Michael-type addition of PEG-dithiol crosslinkers onto 4-arm PEG-Acrylate (4-arm PEG-Ac) in a 0.3 M triethanolamine (TEA) buffer of pH 7-8. The ratio of Ac:SH was kept 1:1 for all conditions. Gelation occurred in several minutes. This gelation strategy is ideal in several ways: 1) highly selective crosslinking chemistry, 2) hydrolytic degradability, 3) ability to incorporate therapeutics or contrast during gelation as well as loading post-fabrication, 4) homogeneous nanoporous structure, and 5) amenability to bead fabrication.

PEG degradation was controlled using a library of custom-synthesized PEG-based dithiol crosslinkers that are biocompatible and biodegradable to achieve degradation times of hours to months (Table 1). Degradation can also be efficiently controlled by buffer pH, PEG molecular weight, PEG precursor concentration, number of PEG-Ac arms, and crosslinker identity.

Electrospraying reliably produced properly-sized microspheres. For embolization, microspheres having a diameter of 100-300 μm were prepared using a PEG precursor solution prepared from 4-arm PEG-Ac and PEG-dithiol crosslinkers described above. The solution was transferred to a hypodermic syringe connected to a needle. The electrospraying setup consisted of a mechanical syringe pump, a direct current high voltage generator connected to the needle tip, and a grounded collector. Solution droplets were collected in a grounded oil bath, allowed to gel for 20 minutes, collected by centrifugation, and washed with PBS to remove oil. Microspheres were obtained having diameters from 50 to 400 μm and <15% coefficient of variance by varying voltage, flow rate, needle gauge, and distance of needle from the oil bath. Importantly, spherical shape was retained upon injection through a 28G needle. Microspheres could be stored hydrated at 4° C. or −80° C. for up to 7 days and lyophilized for 28 days or longer, without significant changes in physical or mechanical properties.

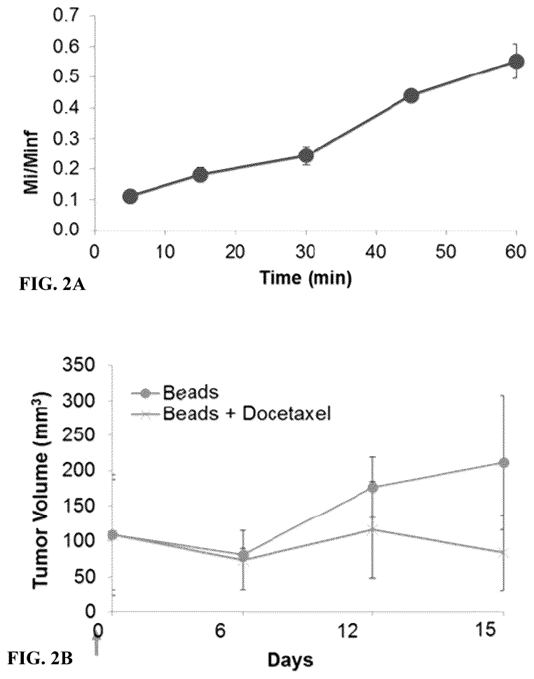

Docetaxel release and in vivo efficacy. Docetaxel was released from the PEG microspheres over several hours, with 60% released in the first 60 minutes (FIG. 2A). For bulk release experiments, docetaxel was dissolved in anhydrous ethanol, loaded in PEG gels at 0.1 mg/ml and released in a 20:80 anhydrous ethanol:di-water. Release was followed by absorbance measurements at 220 nm. Docetaxel-loaded beads reduced the volume of PC3 cells in a mouse xenograft, while beads alone did not (FIG. 2B). PC3 cells (2×10⁶ cells) were injected and grown to ˜120 mm³ tumor volume prior to treatment.

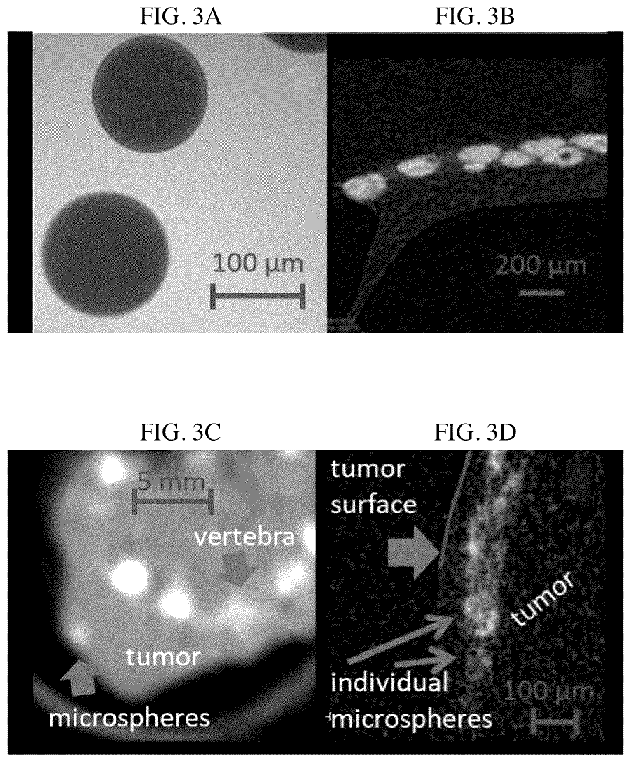

Imageable microspheres were produced by the addition of barium sulfate to PEG microspheres. FIGS. 3A and 3B show microscope and microcomputed tomography (microCT) images of PEG beads containing barium sulfate. An initial in vivo experiment was also performed to assess the imageability of the barium sulfate-loaded beads (FIGS. 3C and 3D). Beads were injected directly into the tumor tissue of a mouse with a xenograft PCa tumor. The mouse was sacrificed and imaged (whole) with clinical cone beam CT (Artis, Siemens, Erlangen Germany). The tumor was then removed and imaged in a microCT scanner (Scanco Medical, Wayne, PA). The results showed that a relatively small number of spheres can be imaged and localized with a clinical cone beam CT scanner, which is available on the angiography system used for embolization in humans.

### Example 2

### Materials

4-arm polyethylene glycol acrylate (4-arm PEG-Ac) with a molecular weight of 10 kDa and polyethylene glycol dithiol (PEG-diSH) with a molecular weight of 3.4 kDa were purchased from Laysan Bio (Arab, AL). 1 μm and 0.1 μm barium sulfate particles and bovine serum albumin (BSA) were purchased from Millipore Sigma (St. Louis, MO). Triethanolamine (TEA) was purchased from Thermo Fisher Scientific (Waltham, MA). Laponite XLG, also known as nanosilicates (NS) were purchased from BYK Additives (Wesel, Germany). Phosphate buffer saline (PBS) was purchased from Mediatech Inc. (Manasssas, VA). Docetaxel was purchased from Biotang Inc. (Lexington, MA). Bicalutamide was purchased from Tokyo Chemical Industry (Portland, OR). Apalutamide was purchased from MedChemExpress (Monmouth Junction, NJ). Gas-tight syringes, 100 l and 25 mL, were purchased from Hamilton Company (Reno, NV). T-junction with a bore diameter of 0.5 mm and polytetrafluoroethylene (PTFE) tubing (inner diameter of 0.5 mm) were purchased from Valco Instruments Co. Inc. (Houston, TX). Female luer locks ( 1/16″) were obtained from Idex Health & Science (Westbrook, ME). Glass plate slides (10.00×8.25×1 cm³) were purchased from Bio-Rad Laboratories (Hercules, CA). Silicone spacers were purchased from Grace Bio-Labs (Bend, Oregon). Programable syringe micropumps were purchased from New Era Pump Systems Inc. (Farmingdale, NY). Kimwipes were purchased from Kimberly-Clark Professional (Roswell, GA). Olive oil was purchased from a local grocery store. Steel rod (⅝-inch×3 feet) and donepart bearings (⅝-inch) were purchased from Amazon. Servomotor (RC 120 RPM 6V Parallax Inc.) was from Digi-key Electronics.

### Fabrication of Hydrogels

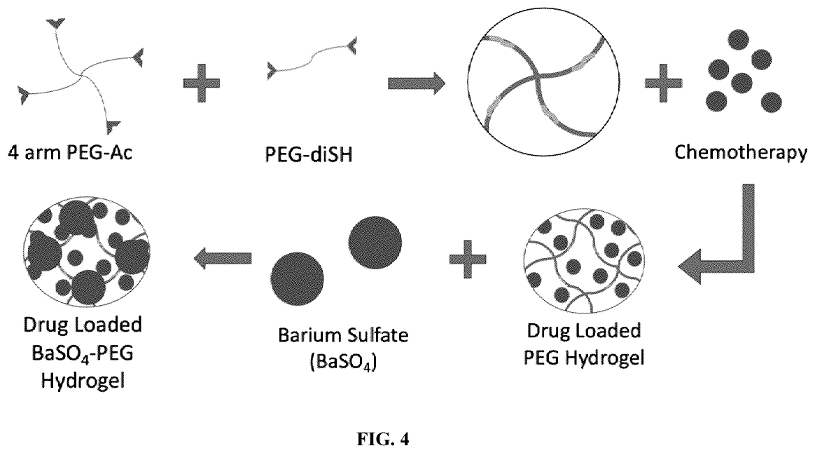

Hydrogels were fabricated using 4-arm PEG Ac and PEG-diSH. These two polymers form a crosslinking network that encapsulates the contrast agent, nanomaterials and drugs as shown in FIG. 4. Stock solutions of 4-arm PEG-Ac and PEG-diSH with a concentration of 20% w/v were prepared by dissolving both polymers in a 0.3 M TEA buffer with a pH of 7.4. To prepare the final solution, 29.8 μL and 20.2 μL of 4-arm PEG-Ac and PEG-diSH, respectively were used. 10 μL of barium sulfate stock and 40 μL of TEA is the remaining solution used to make 20% w/v PEG gels. Barium sulfate particles (10% w/v final concentration) and BSA (5% w/v final concentration) were mixed in PBS to prepare a barium sulfate stock solution. The barium sulfate stock solution was dispersed using a sonicator (Sonic Dismembrator Model 100, Thermo Fisher Scientific, Waltham, MA, USA) for 5 min (1 sec on/1 sec off). Barium sulfate stock was then added to the 4-arm PEG-Ac. TEA was added next to the precursor solution. Lastly, PEG-diSH was then added and mixed thoroughly. Immediately after, three 30 μL volumes were dispensed on a glass plate with 2 mm spacers in the four corners of the plate. Another glass plate was placed on top and pressed down to ensure the spacers were in contact with the top glass plate. An hour and thirty minutes were timed to ensure gelation and then the top plate was removed to retrieve the hydrogel slabs.

### Bulk Release of Chemotherapeutic

To determine bulk release of drugs from hydrogels, acridine orange (AO), docetaxel, bicalutamide, and apalutamide release into a sink volume was measured. Samples from the sink volume were taken over time and then the amount of sample volume that was taken out was replaced with the same amount of sink solution. The sink solution for AO experiments was PBS and the sink solution used for docetaxel, bicalutamide, and apalutamide was 80:20 ethanol to DI water. The absorbance of each sample solution was measured to calculate the concentration of drug in the sample solution. The equation used to calculate these concentrations was from the standard curves obtained for each drug or acridine orange (AO). The concentrations of drugs or AO in solution allowed for quantifying each drug release profile as well as their diffusion coefficient from a hydrogel.

### Acridine Orange (AO) Release

A standard curve for acridine orange (AO) was created initially by measuring the fluorescence of known concentrations of AO, which allowed for the calculation of AO concentration based on absorbance measurements obtained from the samples. AO replaced a portion of TEA during loading to maintain a total volume of 100 μL. Four conditions of gels were prepared, namely PEG only, PEG+5% BaSO₄, PEG+10% BaSO₄, and PEG+20% BaSO₄ (Table 2).

TABLE 2
Volumes (in μL) used to make hydrogels with AO.

| 3 × 30 μL Gels | 0% | 1% | 10% | 20% |
|---|---|---|---|---|
| (100 μL solution) | BaSO₄ | BaSO₄ | BaSO₄ | BaSO₄ |
| 4-arm PEG-Ac | 29.8 | 29.8 | 29.8 | 29.8 |
| (20% w/v) | | | | |
| PEG-diSH (20% w/v) | 20.2 | 20.2 | 20.2 | 20.2 |
| TEA (0.3M, pH 7.4) | 40 | 40 | 40 | 40 |
| Acridine Orange | 10 | 10 | 10 | 10 |
| (0.1 mM) | | | | |
| Barium Sulfate (mg) | 0 | 1 | 10 | 20 |
| BSA (mg) | 5 | 5 | 5 | 5 |

The same method for gelation described above was used for these four types of gels. Three 30 μL gels were created for each condition. After gelation, each gel was placed in a microfuge tube containing 1000 μL of PBS. Time points were taken at 10 min, 30 min, 1 hr, 4 hr, 1 day, 3 day, 6 day, and 7 day intervals. At each time point, 200 μL samples of PBS were taken from each microfuge tube and replaced with 200 μL of fresh PBS. Each sample's fluorescence was measured using a spectrophotometer at 540 nm. The fluorescence values were divided by 2, using the standard curve equation for AO, to calculate the concentration of AO in solution. The concentration was then multiplied by 1, the sink volume (1 mL), which calculates the mass of AO that has been released. Cumulative mass for the next time points were calculated the same way and then added to the mass of the previous time points. Equation 2.1 is how the cumulative mass released was calculated for each time interval. Mi is the concentration of released solute at time I, Ci is the concentration of solute in solution at time I, V is the total volume of released solution (1 mL), and Vs is the sample volume (0.20 mL). This lets us measure the cumulative mass release of AO into solution over time. Fractional release can then be calculated by dividing the cumulative mass released at each time point by the maximum average cumulative mass released from the gels.

$${M}_{i} = {C}_{i} V + \sum {C}_{i - 1} {V}_{s} \qquad Equation 2.1$$

The fractional release was calculated using Equation 2.2. Fi is the fractional amount of solute release at time i.

$${F}_{i} = \frac{{M}_{i}}{{M}_{max}} \qquad Equation 2.2$$

Lastly, a modified form of Fick's law for short release times was used to calculate the effective diffusion coefficient, as described in Equation 2.3.

$$\frac{{M}_{i}}{{M}_{i n f}} ≅ {2 [ \frac{{D}_{e} t}{π {δ}^{2}} ]}^{1 / 2} \qquad Equation 2.3$$

### Absorbance Scans

Drug solutions were prepared for each scan using a mixture of 80:20 ethanol to DI water. The drugs, with a concentration of 0.5 mg/mL, were dissolved in DMSO. Subsequently, each solution was transferred to a glass cuvette and inserted into a spectrophotometer to obtain an absorbance scan ranging from 200 nm to 400 nm, with a step size of 1 nm. The obtained data was then plotted to determine the absorbance of each drug. The calculations for the solutions are summarized in Table 3.

TABLE 3
Volumes (in μL) used for absorbance scan of drugs.

| | Volume of | Volume of | Volume of DI |
|---|---|---|---|
| | drug (μL) | ethanol (μL) | water (μL) |
| Bicalutamide | 5 | 396 | 99 |
| (0.5 mg/mL) | | | |
| in DMSO | | | |
| Docetaxel | 5 | 396 | 99 |
| (0.5 mg/mL) | | | |
| in DMSO | | | |
| Apalutamide | 20 | 384 | 94 |
| (0.5 mg/mL) | | | |
| in DMSO | | | |

### Docetaxel Release

Docetaxel (0.5 mg/mL) stock solution was prepared in 80:20 DMSO or ethanol and DI water. Known concentrations of docetaxel in 80:20 DMSO or ethanol to DI water were prepared, and absorbances were measured using a spectrophotometer at 230 nm to create a standard curve for docetaxel concentration. The same method for fabricating the hydrogels was used, replacing NS and AO with docetaxel. The volume of docetaxel used was subtracted from the TEA volume to maintain the total precursor solution volume at 100 μL. Three conditions were tested: PEG only, PEG+Docetaxel, and PEG+20% BaSO₄+Docetaxel (Table 4). Each gel was placed in a microfuge tube with 750 μL of 80:20 ethanol to DI water. Samples of 500 μL were taken out and replaced with 500 μL of fresh 80:20 ethanol to DI water. The samples were placed in a 96-well plate and absorbances were read using a spectrophotometer at 230 nm. These absorbances were plugged into the standard curve equation to calculate cumulative mass and fractional release.

TABLE 4
Volumes (in μL) used for fabricating
hydrogels containing NS and docetaxel.

| | PEG- | PEG + | PEG + | PEG + |
|---|---|---|---|---|
| 3 × 30 μL gels each | Only | NS | BaSO₄ | BaSO₄ + NS |
| 20% 4-arm PEG-Ac | 29.8 | 29.8 | 29.8 | 29.8 |
| 20% PEG-diSH | 20.2 | 20.2 | 20.2 | 20.2 |
| Docetaxel (0.5 mg/mL) | 30 | 30 | 30 | 30 |
| 20% BaSO₄ | 0 | 0 | 10 | 10 |
| 0.3M TEA | 20 | 10 | 10 | 0 |

Bicalutamide and Docetaxel Release with Solvents DMSO and Ethanol

Bicalutamide (0.5 mg/mL) stock solution was prepared in 80:20 DMSO or ethanol and DI water. A standard curve for bicalutamide was obtained by measuring the absorbance at 270 nm of known concentrations of bicalutamide in 80:20 DMSO or ethanol to DI water. Two types of gels were fabricated: PEG+Doc+Bic and PEG+BaSO₄+Doc+Bic (Table 5). Three gels were made for each condition and then placed in a microfuge tube with 1 mL of 80:20 ethanol to DI water. Time points of 10 min, 30 min, 1 hr, 2 hr, 4 hr, 6 hr, 1 day, 2 day, 3 day, and 7 day were taken. Absorbances at 230 nm and 270 nm were measured for each sample to determine the concentrations of docetaxel and bicalutamide released into the solution.

TABLE 5
Volumes (in μL) used for fabricating hydrogels
with bicalutamide and docetaxel.

| | | PEG + | PEG + | PEG + |
|---|---|---|---|---|
| | PEG + | BaSO₄ + | Bic + | BaSO₄ + |
| 3 × 30 μL | Doc + Bic | Doc + Bic | Doc | Bic + Doc |
| gels each | (Ethanol) | (Ethanol) | (DMSO) | (DMSO) |
| 20% 4-arm PEG-Ac | 29.8 | 29.8 | 29.8 | 29.8 |
| 20% PEG-diSH | 20.2 | 20.2 | 20.2 | 20.2 |
| Docetaxel (0.5 | 20 | 20 | 0 | 0 |
| mg/mL) in ethanol | | | | |
| Bicalutamide (0.5 | 20 | 20 | 0 | 0 |
| mg/mL) in ethanol | | | | |
| 20% BaSO₄ | 0 | 10 | 0 | 10 |
| 0.3M TEA | 10 | 0 | 10 | 0 |
| Docetaxel (0.5 | 0 | 0 | 20 | 20 |
| mg/mL) in DMSO | | | | |
| Bicalutamide (0.5 | 0 | 0 | 20 | 20 |
| mg/mL) in DMSO | | | | |

### Apalutamide and Docetaxel Release

Apalutamide (0.5 mg/mL) stock solution was prepared in 80:20 DMSO or ethanol and DI water. A standard curve was constructed for apalutamide by creating samples of known concentrations and measuring the absorbance at 260 nm of each sample. Hydrogels were then fabricated with conditions: PEG+Doc+Apa and PEG+BaSO4+Doc+Apa (Table 6). The hydrogels gelled over a period of one hour and thirty minutes and were then placed into microfuge tubes with 80:20 ethanol to DI water. Time points of 10 min, 30 min, 1 hr, 2 hr, 4 hr, 6 hr, 1 day, 2 day, 3 day, and 7 day were taken. Absorbances of 230 nm and 260 nm were measured for each sample to be able to obtain concentrations of docetaxel and apalutamide released into solution.

TABLE 6
Calculations for fabricating hydrogels
containing apalutamide and docetaxel.

| | PEG + Doc + | PEG + BaSO₄ + |
|---|---|---|
| 3 x 30 μL gels each | Apa | Doc + Apa |
| 20% 4-arm PEG-Ac | 29.8 | 29.8 |
| 20% PEG-diSH | 20.2 | 20.2 |
| Docetaxel (0.5 mg/mL) | 20 | 20 |
| Apalutamide (0.5 mg/mL) | 20 | 20 |
| 20% BaSO₄ | 0 | 10 |
| 0.3M TEA | 10 | 0 |

### Radiopaque Microsphere Fabrication and Characterization

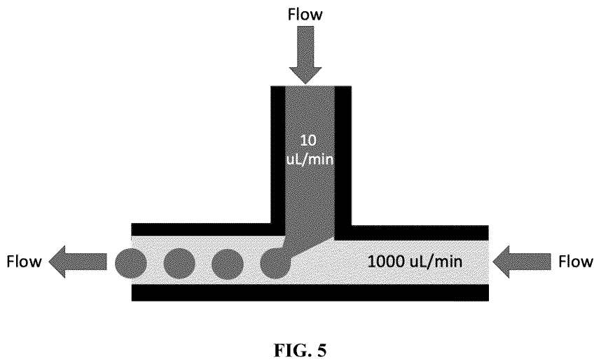

Hydrogel precursor solutions was prepared as described above and used to fabricate hydrogel microspheres. A T-junction was used to create the microspheres and this process is illustrated in FIG. 5. 20% 4-arm PEG-Ac and PEG-diSH were created by dissolving the mass weighed out in the required volume of TEA. TEA (0.3 M, pH 7.4) was used as the buffer. A 20% w/v barium sulfate stock solution was then prepared and mixed with the 4-arm PEG-Ac. Docetaxel and apalutamide (0.5 mg/mL) were then prepared. Two conditions of microspheres were made: PEG+BaSO₄ and PEG+BaSO₄+Doc+Apa. Volumes used are summarized in Table 7.

TABLE 7
Volumes (in μL) used for fabricating
hydrogels with 20% BaSO₄ and drugs.

| | PEG + | PEG + BaSO₄ + |
|---|---|---|
| 300 μL solutions | BaSO₄ | Doc + Apa |
| 20% 4-arm PEG-Ac | 89.4 | 89.4 |
| 20% PEG-diSH | 60.6 | 60.6 |
| Docetaxel (0.5 mg/mL) | 0 | 60 |
| Apalutamide (0.5 mg/mL) | 0 | 60 |
| 20% BaSO₄ | 30 | 30 |
| 0.3M TEA | 120 | 0 |

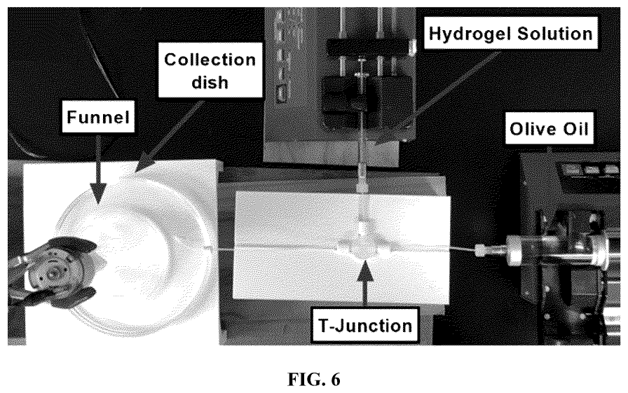

After mixing the components for either condition, the gel precursor solution was drawn up into a 100 μL syringe (10 mm) and attached to a female luer lock connected to tubing and to a T-junction. The system used contained two gas-tight syringes, a T-junction, tubing, 1/16″ female luer locks, a glass collection disk, and two programmable syringe micropumps (NE4002-×, NE-300, New Era Pump Systems Inc., Farmingdale, NY). The syringe was then placed into one of the micropumps to be injected at a rate of 10 μL/min. The other inlet to the T-junction was a 25 mL syringe full of olive oil in another micropump being injected at 800 μL/min. The outlet of the T-junction had an open tube leading to a dish filled with olive oil for the microspheres to be collected. An inverted glass cylinder connected to a DC motor being held by a ring stand was used to stir the olive oil to prevent droplet aggregation. The microsphere device used for fabrication is illustrated in FIG. 6.

Microspheres were imaged using an inverted microscope (Axiovert 200, Zeiss) and analyzed for size with Image J (downloaded free from imagej.nih.gov). Each image had a scale bar placed in the bottom right to measure spheres accurately. The diameter of the microsphere was calculated using a proportion of the pixel length of a microsphere, representing the diameter, to the pixel length of the scale bar. This is how all microsphere sizes pre-swelling and post-swelling were measured.

### Microsphere Release Study

Barium microspheres (20% w/v) were fabricated using the T-junction and then frozen overnight at −80 degrees Celsius. The next morning the microspheres were lyophilized for 1 day. Once the microspheres were fully lyophilized, they were then swollen in 10 μL 0.5 mg/mL docetaxel and apalutamide for 1 day. The next day, the excess drug was removed from the centrifuge tube and 500 μL 80:20 ethanol to DI water was added. A release study was then preformed as described above and the release profile for 20% barium microspheres was obtained.

### Rheology

300 μL barium hydrogels with and without drugs were fabricated using previous methods mentioned. Both gels were swollen overnight in PBS. Excess PBS was removed from the hydrogels by dabbing them a KimWipe. A time sweep analysis was then conducted on both barium hydrogels, applying a strain of 1% and angular frequencies ranging from 1 to 10 rads/s, with 6 data points per decade. This allowed for the determination of the storage modulus (G′) and loss modulus (G″), representing the materials' ability to store and dissipate energy, respectively.

### Statistical Analysis

All results are averages ±standard deviation of three independent experiments with three samples for each group (n=9). A one-way ANOVA was used to determine statistical significance between all samples. Two-tailed student's t-test was also used to determine statistical significance between two samples. p<0.05 was the value considered to make the data significant.

### Results

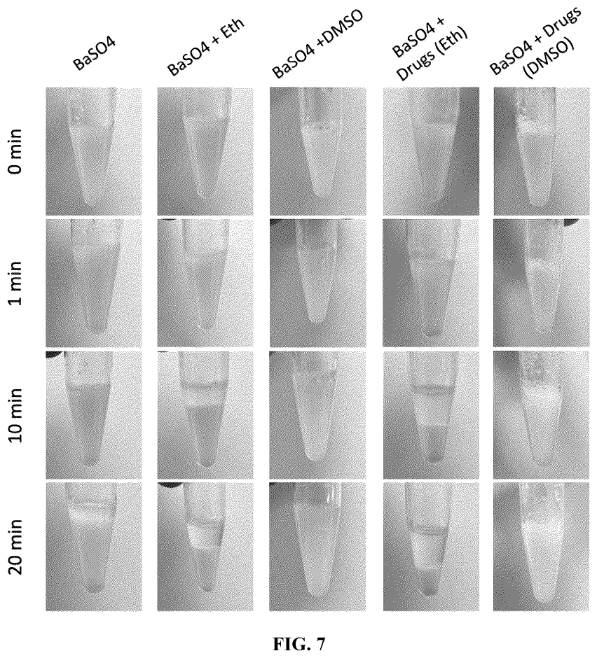

As shown in FIG. 7, different solvents for drugs, namely ethanol and DMSO with and without drugs (docetaxel and bicalutamide), were tested to determine the effects on the settling of barium sulfate. Stable barium sulfate suspension was needed to prepare imageable microspheres with evenly dispersed imaging agent. BaSO₄ was the control hydrogel group that used TEA (0.3 M in PBS, pH 7.4) as the solvent. This group was the control for how barium acts normally in a hydrogel. At the 10 min time point, barium sulfate in both ethanol groups (with and without drugs) began to settle in solution. By 20 mins both ethanol groups settled even more. The BaSO₄ (in TEA) control group began to settle very slightly by 20 mins, but was not as rapid as the two ethanol groups. The groups containing DMSO also began settling by 20 mins. DMSO did not accelerate the settling of barium sulfate as fast as ethanol and kept the barium sulfate suspended longer than the BaSO₄ group.

### Imageability of Hydrogels

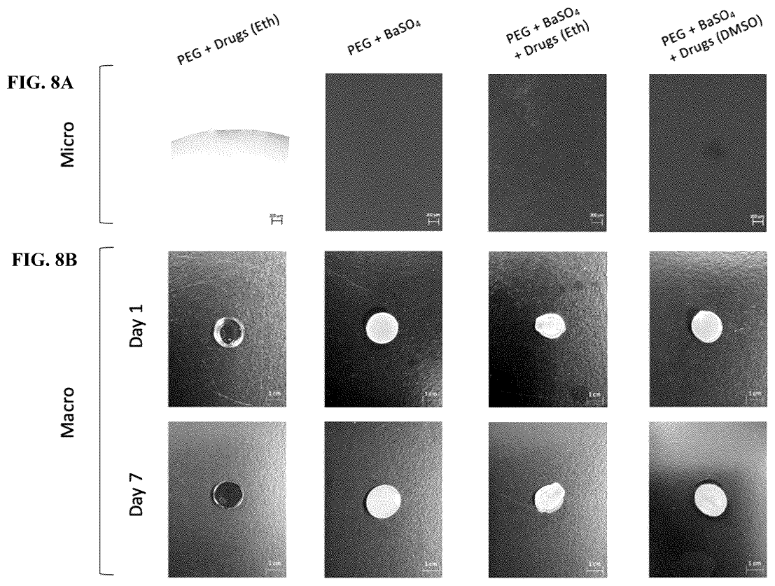

FIG. 8A shows microscale images of hydrogels. In FIG. 8A, the PEG +Bic+Doc group appeared clear like a PEG hydrogel. The PEG+BaSO₄group depicts a hydrogel with suspended barium sulfate. The two groups including ethanol and DMSO were compared to the opaqueness of the PEG+BaSO₄ group. PEG+BaSO₄+Ethanol showed patches of clear space meaning barium sulfate settled during the gelation process. Therefore, ethanol mitigated suspension of barium sulfate and ultimately decreased the imageability of the hydrogel. The PEG+BaSO₄+DMSO group was similar to the PEG+BaSO₄ group indicating it had little effect on the suspension of barium sulfate and subsequent imageability. The addition of drugs dissolved in DMSO did not influence imageability.

FIG. 8A shows images of the hydrogels on a macroscale on day 1 and day 7. FIG. 8B shows imageability after gelation and swelling. PEG+Bic+Doc was clear because PEG forms a clear gel and the chemotherapy drugs are colorless. PEG+BaSO₄ hydrogels had a consistent suspension of barium throughout the gel. PEG+BaSO₄+Drugs (Ethanol) hydrogels had inconsistent dispersion of barium sulfate because of the clear patches observed in the hydrogel. PEG+BaSO₄+Drugs (DMSO) most closely resembled the PEG+BaSO₄ hydrogels visually. The barium sulfate in both had consistent suspension with no clear patches.

### Effect of Drug Encapsulation on Microsphere Opacity, Size, and Swelling

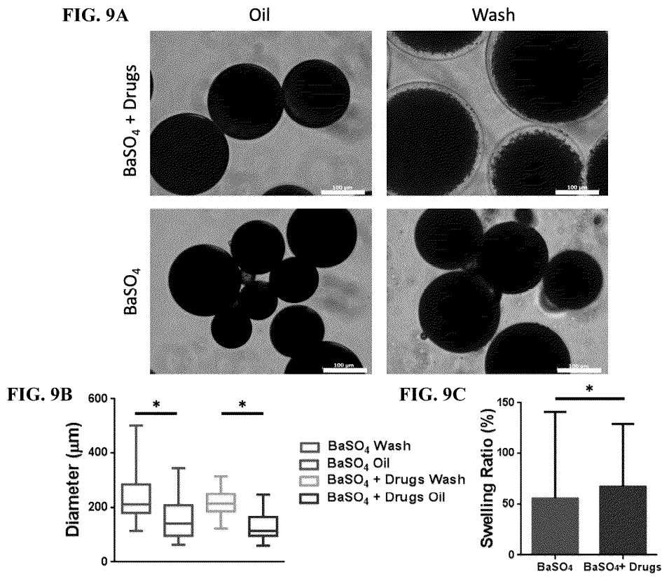

FIG. 9A shows imageability of barium sulfate when encapsulated in microspheres and the effects the chemotherapy drugs, when dissolved in DMSO, have on the imageability. This figure also shows what washing the microspheres does to the imageability. After washing it is seen that the microspheres swell and lose some of the imageability because of the increase in volume. Most importantly our results show that DMSO does not affect imageability of barium sulfate when encapsulated in microspheres. FIG. 9B shows a histogram of the diameter of 1 μm BaSO₄ microspheres with and without drugs loaded. Both groups have the same distribution of diameter meaning the addition of drugs does not affect size or swelling of the microspheres. FIG. 9C shows the swelling ratio of the two groups, 1 μm BaSO₄ with and without drugs. The swelling ratio data is not significant and is neglected meaning both groups have very similar swelling capabilities.

### Effect of Barium Sulfate Particle Size on Microsphere Opacity, Size, and Swelling

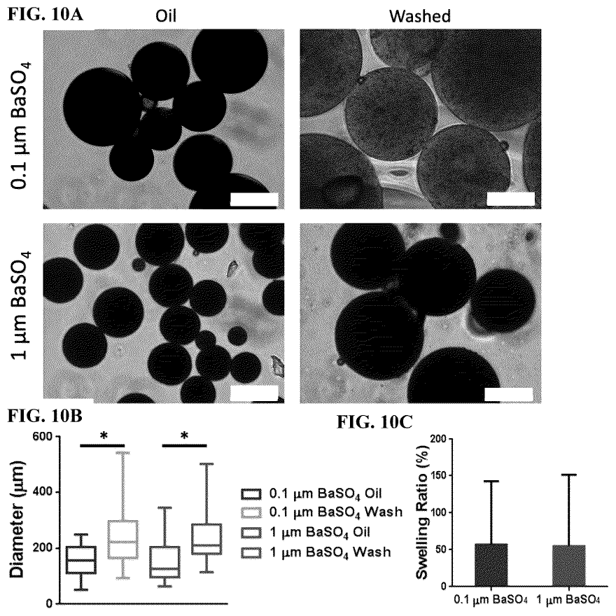

The effect of BaSO₄ particle size, namely 0.1 vs 1 μm, on microsphere properties was determined. The effects of BaSO₄ particle size on microspheres are depicted in FIG. 10A. As particle size increased, the average microsphere size decreased. The 1 μm BaSO₄ spheres did not lose their opacity after swelling. Larger size BaSO₄ particles were found to be better for imageability and were used for further experiments. The quantitative data of microspheres containing different sizes of BaSO₄ particles is depicted in FIG. 10B. The average size of microspheres containing 1 μm BaSO₄ trended smaller than the average size of 0.1 μm BaSO₄ spheres. However, the data is not significant for the sphere size in olive oil, indicating that the size difference was negligible. When swelled, the average sphere size for 1 μm BaSO₄ microspheres trended larger. The data shows that spheres with larger BaSO₄ particle size had greater swelling (FIG. 10C). 1 μm BaSO₄ spheres swell almost 50% of their original size while 0.1 μm BaSO₄ spheres swell around 25% of their original size.

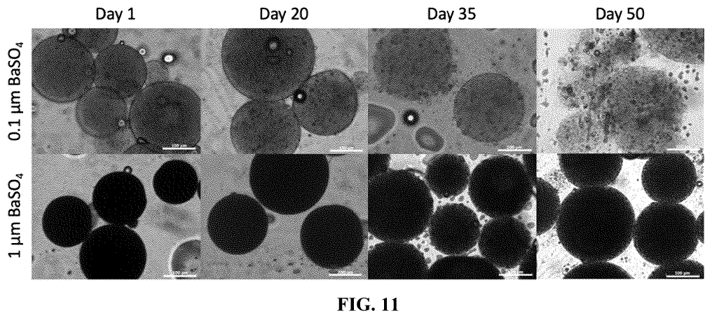

Microspheres were imaged over a course of 35 days to determine imageability and degradation. Degradation for both groups over this duration of time was relatively the same indicating that barium sulfate did not affect the degradation rate of the microspheres. As shown in FIG. 11, 1 μm BaSO₄ particles made the microspheres more opaque compared to 0.1 μm BaSO₄ particles. After 35 days, no significant decrease in imageability was observed for microspheres containing 1 μm BaSO₄ particles.

### Absorbance Scans for Drugs

For each drug, the absorbance wavelength was determined by performing an absorbance scan of a solution with drug from 200 nm to 400 nm. Peaks for docetaxel, bicalutamide, and apalutamide were observed at 230 nm, 260 nm, and 270 nm, respectively.

### Bicalutamide and Docetaxel Release

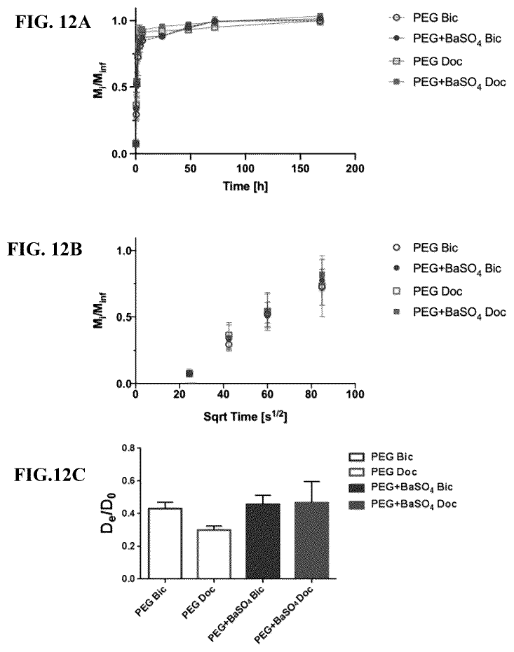

FIG. 12A depicts data for the cumulative mass release of bicalutamide and docetaxel individually and as a function of barium sulfate loading. Results show that the addition of barium sulfate, despite being larger than the meshwork of the hydrogel, did not alter the release kinetics. There was a large burst release for both drugs within the first 24 h. Results show around >80% of their mass was released during this period. The fractional release is depicted in FIG. 12B and is plotted against the square root of time. The data for the fractional release depicted in FIG. 12B shows that the mass of bicalutamide and docetaxel released were very similar, indicating that they have very similar diffusion coefficients (see, FIG. 12C). These results indicate that both drugs can be loaded effectively in an imageable PEG hydrogel that contains barium sulfate and that barium sulfate does not affect drug release. The drugs were released with a burst release due to the smaller size compared to the mesh size of the PEG hydrogel.

### Apalutamide and Docetaxel Release

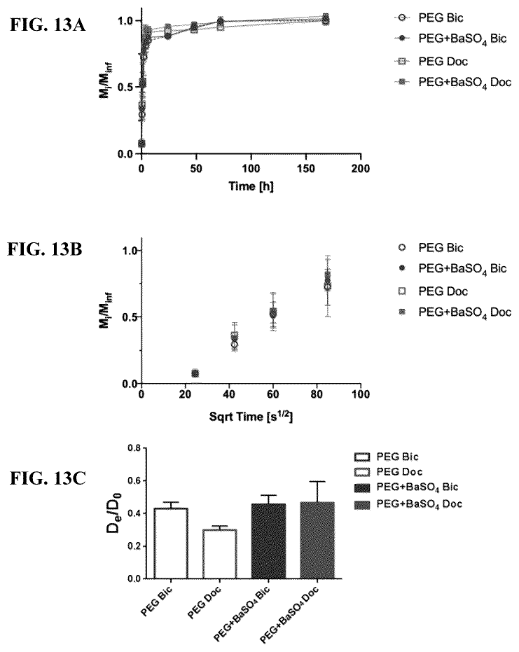

The release of docetaxel and apalutamide from the same 20% BaSO₄ hydrogel is depicted in FIGS. 13A-13C. Docetaxel and apalutamide were observed to have very similar releases from both a PEG-only and 20% BaSO₄ hydrogel. Both docetaxel and apalutamide were released at very similar rates. After 24 h nearly 90% of the docetaxel was released from both hydrogel groups. The effective diffusion coefficients were also very similar for both groups, showing almost equal diffusion coefficients for both docetaxel and apalutamide.

### Apalutamide and Docetaxel Microsphere Release

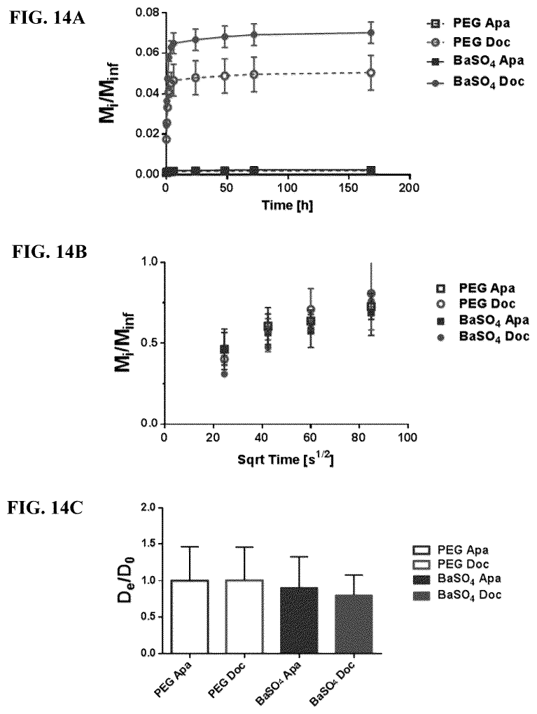

FIGS. 14A-14C demonstrate the release of apalutamide and docetaxel from microspheres containing fully suspended 1 μm BaSO₄ at a concentration of 20% w/v. Apalutamide and docetaxel had a similar release in both the PEG and BaSO₄ groups. Docetaxel in both groups had an initial burst release of ˜90% within the first 24 hrs. Apalutamide release was around ˜80% within the first 24 hrs. Diffusion coefficients for apalutamide and docetaxel were also very similar but showed that the average diffusion coefficients for the BaSO₄ groups were slower. This data confirmed that, similar to slab hydrogels, release of the drugs from the imageable microspheres was not affected by BaSO₄.

### Effect of Drug on Storage and Loss Modulus

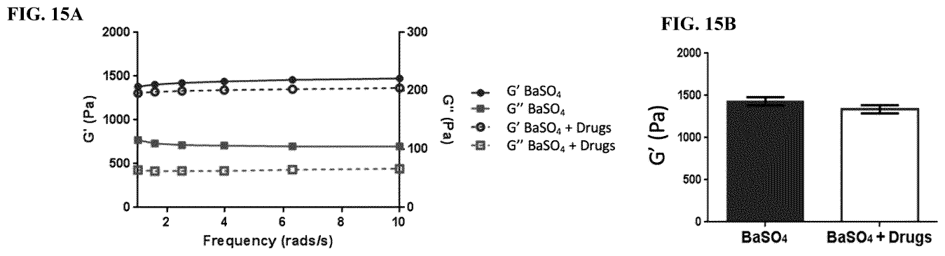

Experiments were conducted to determine whether drugs affect the storage and loss modulus of a barium sulfate-loaded hydrogel. Minimal decrease in the storage and loss modulus for barium sulfate hydrogels loaded with drug were observed (FIGS. 15A and 15B). This indicated that the addition of drugs made the gels slightly less stiff.

As disclosed herein, bead embolization can safely treat prostate tumors. Prostate tumors will shrink—with minimal unwanted side effects—if microspheres loaded with chemodrugs and/or hormones are precisely placed in nearby arteries. The microspheres block blood from entering and feeding the tumor, while simultaneously delivering and trapping anti-cancer drugs and hormones inside the tumor. The microspheres: 1) contain and release appropriate drugs and/or hormones; 2) can be seen by x-rays, to confirm precise delivery to the tumor; and 3) can be safely resorbed into the body, for repeat treatment. This presents a unique, comprehensive approach, integrating targeted embolization, chemotherapy and hormone therapy as a minimally invasive and repeatable treatment.

In view of the above, it will be seen that the several advantages of the disclosure are achieved and other advantageous results attained. As various changes could be made in the above methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. An embolization composition comprising:
a plurality of biocompatible and resorbable polymer hydrogel microspheres comprising a contrast agent; a hormonal therapy; and a chemotherapeutic; and
a carrier.

2. The embolization composition of claim 1, wherein the biocompatible and resorbable polymer hydrogel microsphere comprises a polymer selected from the group consisting of poly(ethylene glycol)-di-acrylate (PEG-DA), multi-arm poly(ethylene glycol)-acrylate (PEG-Ac), poly(ethylene glycol) divinyl sulfone (PEG-diVS), multi-arm poly(ethylene glycol) vinyl sulfone (PEG-VS), poly(ethylene glycol)-di-methacrylate (PEG-DMA), multi-arm poly(ethylene glycol)-methacrylate (PEG-MAc), poly(ethylene glycol)-di-allyl ether (PEG-diAE), multi-arm poly(ethylene glycol)-allyl ether (PEG-AE), poly(ethylene glycol)-di-vinyl ether (PEG-diVE), multi-arm poly(ethylene glycol)-vinyl ether (PEG-VE), poly(ethylene glycol)-di-maleimide (PEG-diMI), multi-arm poly(ethylene glycol)-maleimide (PEG-MI), poly(ethylene glycol)-di-norborene, multi-arm poly(ethylene glycol)-norborene, poly(ethylene glycol)-di-vinyl carbonate, multi-arm poly(ethylene glycol)-vinyl carbonate, polyethylene glycol oligofumarate, and combinations thereof.

3. The embolization composition of claim 1, wherein the biocompatible and resorbable polymer hydrogel microsphere further comprises a crosslinker selected from the group consisting of a thiol-functionalized molecule, a multi-arm polyethylene thiol (PEG-SH), a degradable poly(ethylene glycol)dithiol-based crosslinker, dithiothreitol (DTT), glycol dimercaptoacetate (GDMA), glyceryldithioglycolate (GDT), glycol di(3-mercaptopropionate) (GDMP), tetraethylene glycol dithiol (TEGDT), 2,2′-(ethylenedioxy) diethanethiol (EDDT), 2-amino butane dithiol (DTBA), and combinations thereof.

4. The embolization composition of claim 1, wherein the contrast agent is selected from the group consisting of barium sulfate, iodine, gold, zirconium oxide, bismuth, and combinations thereof.

5. The embolization composition of claim 1, wherein the hormonal therapy is an androgen receptor antagonist.

6. The embolization composition of claim 5, wherein the androgen receptor antagonist is selected from the group consisting of bicalutamide, apalutamide, darolutamide, enzalutamide, flutamide, nilutamide, cyproterone acetate, medroxyprogesterone acetate, megestrol acetate, and combinations thereof.

7. The embolization composition of claim 1, wherein the chemotherapeutic is selected from the group consisting of docetaxel, cabazitaxel, mitoxantrone, estramustine, and combinations thereof.

8. The embolization composition of claim 1, wherein the biocompatible and resorbable polymer hydrogel microspheres have an average diameter ranging from about 50 μm to about 700 μm.

9. The embolization composition of claim 1, wherein the biocompatible and resorbable polymer hydrogel microspheres degrade at a rate ranging from about 6 hours to about 8 weeks.

10. The embolization composition of claim 1, wherein the hormone therapy is released from the biocompatible and resorbable polymer hydrogel microspheres at a rate ranging from about 5 minutes to about one week.

11. The embolization composition of claim 1, wherein the chemotherapeutic agent is released from the biocompatible and resorbable polymer hydrogel microsphere at a rate ranging from about 5 minutes to about one week.

12. The embolization composition of claim 1, wherein the carrier comprises water, saline, isotonic saline, phosphate buffered saline, sodium acetate, glycylglycine, HEPES (4-(2-hydroxyethyl)-1-piperazineethanesulfonic acid), sodium phosphate, dimethyl sulfoxide, ethanol, thiethanolamine, and combinations thereof.

13. The embolization composition of claim 1, wherein the buffer comprises sodium acetate, acetic acid, or a combination thereof.

14. The chemoembolization composition of 1, for use in a method for treating a solid cancer tumor.

15. The chemoembolization composition of claim 14, wherein the solid cancer tumor comprises prostate cancer. hepatoma, hepatocellular carcinoma (HCC), liver metastasis, cholangiomas, neuroendocrine tumors, GIST liver metastasis, and renal cancer.

16. A method of treating prostate cancer in a subject in need thereof, the method comprising:
injecting into a blood vessel of the subject an embolization composition, the embolization composition comprising:
a plurality of biocompatible and resorbable polymer hydrogel microspheres, the plurality of biocompatible and resorbable polymer hydrogel microspheres comprising: a contrast agent; a hormonal therapy; and a chemotherapeutic; and
a carrier.

17. The method of claim 16, wherein the embolization composition is injected directly into the blood vessel providing blood to a prostate tumor in the prostate of the subject in need thereof.

18. The method of claim 16, further comprising imaging the subject to determine at least one of location of the plurality of biocompatible and resorbable polymer hydrogel microspheres and degradation status of the plurality of biocompatible and resorbable polymer hydrogel microspheres.

19. The method of claim 16, further comprising imaging the subject.

20. The method of claim 19, wherein the imaging is performed using microCT; clinical CT; X-ray; and combinations thereof.
